(19) United States
(12) Patent Application Publication
(10) Pub. No.: US 2007/0202145 A1
(43) Pub. Date: Aug. 30, 2007

(54) Method for incorporation of bioactives into a porous hydrophobic polymer scaffold

(76) Inventors: Ragae Ghabrial, Helmetta, NJ (US); Janet Davis, Branchburg, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/749,192

(22) Filed: Dec. 31, 2003

(51) Int. Cl. A61F 2/02; A61K 38/18; A61K 38/22; A61K 35/12; A61K 39/395; A61K 31/203

(52) U.S. Cl. 424/423; 424/93.7; 514/12; 514/559; 424/130.1

(57) ABSTRACT

This idea represents a method for incorporation of a therapeutic molecule, preferably a protein or a growth factor, into a biodegradable scaffold, specifically one that is made of a foam nonwoven composite. The process utilizes a solvent preferably tertiary butanol to facilitate the infiltration of the particles of the therapeutic agent into the porous matrix of the scaffold. In the case of small molecules, such as a p38 kinase inhibitor, the drug is dissolved directly in sterile filtered t-butanol and a given amount is pipetted aseptically onto the pre-sterilized scaffold. The solution is readily adsorbed into the polymer matrix. The solution is readily frozen to allow minimal interaction with the polymer scaffold thereby protecting the scaffold's internal matrix. The solvent is then aseptically removed by lyophilization.

### BACKGROUND OF THE INVENTION

This invention is generally in the field of medicine and cell culture, and in particular in the area of implantable organs formed on biocompatible artificial matrices.

Loss of organ function can result from congenital defects, injury or disease.

One example of a disease causing loss of organ function is diabetes mellitus or, as it is known more simply, diabetes. Diabetes destroys the insulin-producing beta cells of the pancreas. As a consequence, serum glucose levels rise to high values because glucose cannot enter cells to meet metabolic demands. This inability to properly metabolize blood sugar causes a complex series of early and late-stage symptomologies to develop, beginning for example with hyperglycemia, abnormal hunger and thirst, polyuria, and glycouria then escalating to for example neuropathy, macro-vascular disease, and micro-vascular disease. It is these events manifested as symptoms, one or more of which are diagnosed by a treating physician as diabetes. The underlying cause of these symptoms, however, is the lack of production of insulin which must be corrected to stave off diabetic disease and its complications.

The current method of treatment consists of the exogenous administration of insulin, usually through injection by either needles or a pump, which results in imperfect control of blood sugar levels. The degree of success in averting the complications of diabetes remains controversial. It is taken as a given, however, that to the extent pancreatic organ function can be restored or rejuvenated, better control of insulin and, therefore, blood sugar levels, can be attained. Toward that end, much effort has been expended to develop cellular products and transplantation procedures, devices, and instruments to restore or mimic pancreas function.

A recent and still experimental approach includes the transplantation of islets of Langerhans, containing insulin-producing beta cells, into diabetic patients along with a specific type and amount of pharmaceutical compounds to reduce the host immune reaction to the transplanted insulin-producing islets. The islets are injected into the portal vein of the liver because of the relatively large supply of nutrients and the ability of the liver to remove waste products. Serum glucose appears to be controlled in a more physiological manner using this technique and the progression of complications is thereby slowed. (Ryan E A, Lakey J R, Rajotte R V, Korbutt G S, Kin T, Imes S, Rabinovitch A, Elliott J F, Bigam D, Kneteman N M, Warnock G L, Larsen I, Shapiro A M., Clinical outcomes and insulin secretion after islet transplantation with the Edmonton protocol., Diabetes. 2001 April; 50(4):710-9.)

This transplantation method, however, is not without its drawbacks. Complications may develop in the liver itself which in turn could cause hepatic failure unrelated to the patient's diabetes but, instead, to the transplantation procedure itself.

It has been suggested that if a different transplantation site with sufficient vasculature were available, islets could be transplanted to that site and still mimic the physiology of the pancreas, yet avoid damage to the liver (Y. J. Gu, M. Miyamoto, W. X. Cui, B. Y. Xu, Y. Kawakami, T. Yamasaki, H. Setoyama, N. Kinosita, H. Iwata, Y. Ikada, M. Imamura, and K. Inoue, Effect of Neovascularization-Inducing Bioartificial Pancreas on Survival of Syngeneic Islet Grafts, Transplantation Proceedings, 32, 2494-2495 (2000)). One method of creating such a site involves the use of a scaffold to hold the islets in place while extending the necessary blood vessels to the islets. It has also been contemplated that such scaffolds may be impregnated with pharmaceutical compounds to, for example, encourage an increase in vasculature and/or modulate the immune response that such a transplant may cause (A. N. Balamurugan, Yuanjun Gu, *Yasuhiko Tabata, Masaaki Miyamoto, Wanxing Cui, Hiroshi Hori, Akira Satake, Natsuki Nagata, Wenjing Wang, and Kazutomo Inoue, Bioartificial Pancreas Transplantation at Prevascularized Intermuscular Space: Effect of Angiogenesis Induction on Islet Survival, Pancreas, Vol. 26, No. 3, 2003).

Polymeric matrices, typically in the form of microspheres, rods, sheets or pellets, have been employed for the sustained or controlled release of drug products. A variety of techniques, such as solvent evaporation, spray drying, and emulsification, have been utilized to incorporate active agents into polymer matrices. However, these methods are often not suitable for the incorporation of complex bioactive agents because of the high temperatures used, organic solvent exposure, or interactions with pressurized gas. Alternatively, suitable methods such as porogen leaching or simple adsorption onto polymer scaffolds often cause unavoidable loss of a significant percentage of such bioactive agents during processing, a loss which can be cost prohibitive. Also, most of these methods assume uniform distribution of the bioactive agent across a sheet, a solution or an emulsion, which is then processed further to produce a transplantable scaffold smaller in size and containing a relative portion of the bioactive component. This assumption can be inaccurate and dangerous, especially with potent bioactives such as growth factors where a slight increase in the dose delivered may lead to hazardous side effects or where a reduction in the dose could reduce the expected activity.

Assuming successful incorporation of the bioactive agent into a polymer matrix, another challenge arises in maintaining functional activity during sterilization of such scaffolds. The most common sterilization methods are EtO (Ethylene Oxide), e-beam irradiation, or gamma irradiation. Unfortunately, all of these methods may lead to a partial or full destruction of said bioactive agents. In such cases, further studies are required to assess the byproducts of the degraded molecules and any possible associated harmful side effects.

A number of methods have been developed for drug incorporation into a polymeric scaffold or device. These methods are primarily distinguished based on the sensitivity and release characteristics of each individual drug. The drug can be impregnated within the entire scaffold via an injection technique disclosed in U.S. Pat. No. 5,770,417. In another embodiment, the scaffold can be submerged in a solution containing the drug such that the drug fills the interstices within the scaffold. A scaffold can also be immersed in a solution containing the drug, and the solvent allowed to evaporate, thereby precipitating drug on the surface of the scaffold as disclosed in U.S. Pat. Nos. 5,980,551 and 5,876,452. Also, a drug can be adhered to a scaffold by surface modification of the scaffold to allow better attachment, as achieved using techniques such as plasma irradiation (Kwok, Connie S., Horbett, Thomas A., Ratner, Buddy D., “Design of Infection-resistant Antibiotic-releasing Polymers—Controlled Release of Antibiotics through a Plasma-deposited Thin Film Barrier,” Journal of Controlled Release, Volume 62, pp. 301-311 (1999).)

Another common technique is freeze drying in which the drug or its solution is added to a polymer solution and the solvent is sublimed leaving behind a polymer scaffold with molecules of the drug dispersed within. The success of this technique is influenced by the stability of the drug in the organic solvent used to dissolve the polymer. Unfortunately, the choice of an appropriate organic solvent is very limited when working with protein-based therapeutics due to the sensitivity of their complex three-dimensional structures to strong solvents. Organic solvents influence the activity of the protein by interacting with hydrogen bonds, disulfide bonds, and van Der Waals attractive forces that maintain the unique three-dimensional geometry necessary for protein functionality.

In another technique, a water-oil emulsion is created in which the drug or biologic is dissolved in the aqueous phase and the polymer solution comprises the oil phase. This prevents interaction between the drug and the organic solvent to protect activity of the drug in certain cases.

Typically, though, such scaffolds are manufactured from hydrophobic components, such as polyglycolic acid (PGA)/polycaprolactone (PCL) co-polymers, and the pharmaceutical compounds are relatively hydrophilic proteins and/or growth factors. Because of this type of incompatibility between the scaffold material and the pharmaceutical compound, it is difficult to obtain a homogeneous or otherwise uniform concentration of the pharmaceutical compound throughout the scaffold. This inability to obtain a uniform concentration of the pharmaceutical agent makes it difficult to obtain controlled release of the pharmaceutical compound.

It is therefore an objective of the present invention to disclose a method for impregnating a scaffold with a pharmaceutical agent or compound wherein the pharmaceutical agent or compound is uniformly distributed throughout the scaffold.

It is a further objective of this invention to provide a method for obtaining controlled release of a pharmaceutical agent or compound from a scaffold.

It is a further objective of this invention to provide a scaffold impregnated with a therapeutic compound according to the method provided herein.

### SUMMARY OF THE INVENTION

The present invention provides a method for incorporation of therapeutic molecules, preferably but not limited to a protein or a growth factor, into a biodegradable biocompatible scaffold. The scaffold can be a porous foam or a composite scaffold, where a composite scaffold is composed of fibers encapsulated by and disposed within a porous, polymeric matrix.

The incorporation process utilizes an organic solvent, preferably an alcohol or ether with a relatively high melting point, to facilitate the infiltration of the soluble molecules of the therapeutic agent into the porous matrix of the scaffold.

### BRIEF DESCRIPTION OF THE DRAWINGS

FIG. 1 shows two drops of two solutions of VEGF-121 in PBS after ten seconds.

FIG. 2 shows two drops of two solutions of VEGF-121 in PBS after thirty minutes.

FIG. 3 shows an image of a subcutaneous transplantation site.

FIG. 4 shows a scaffold after removal from a transplantation site.

FIG. 5 shows a hematoxylin & eosin section of a scaffold containing VEGF-121 following 2 wks of implantation in a subcutaneous compartment of a rat.

FIG. 6 shows the activity of VEGF-121 after exposure to t-butanol and lyophilization.

FIG. 7 shows the activity of VEGF-121 after recovery from a scaffold in comparison to a stock solution of VEGF-121.

### DETAILED DESCRIPTION OF THE INVENTION

In accordance with the present invention, there is provided an efficient means for incorporation of a bioactive agent into a hydrophobic biodegradable polymer-based scaffold. It preferably targets, but is not limited to, bioactive molecules that are sensitive to traditional methods of sterilization such as EtO, gamma irradiation, or E-beam irradiation. It can also be employed in cases where the bioactive molecules are sensitive to the fabrication processes of the scaffold. The method is also useful for the incorporation of therapeutics with high affinity for aqueous solvents into polymeric scaffolds prepared from a polymer solution in an organic solvent.

There are three major elements to this invention: (1) the scaffold, (2) the pharmaceutical compound or agent, and (3) the process of incorporating or impregnating the pharmaceutical compound into the scaffold. Each will be discussed below.

### A. The Scaffold

The term scaffold used herein refers to a three-dimensional matrix and is generally used to host cells or small organoids for the purpose of transplantation. The scaffold could be prepared using a variety of techniques known to those experts in the art, such as but not limited to lyophilization, salt leaching or extrusion.

The scaffold is preferably a porous foam or a composite scaffold, where a composite scaffold is composed of fibers encapsulated by and disposed within a porous, polymeric matrix. Preferably, the fibers and matrix are biocompatible.

With a composite scaffold, the fibers encapsulated by a porous matrix are preferably in the form of a non-woven, fibrous mat. Typically such are made by wet-lay or dry-lay fabrication techniques. The polymer foam matrix of a composite scaffold is preferably made by a polymer-solvent phase separation technique, such as lyophilization.

The scaffold can be prepared from a variety of polymers, such as but not limited to aliphatic polyesters, which can be homopolymers or copolymers (random, block, segmented, tapered blocks, graft, triblock, etc.) having a linear, branched or star structure. Suitable monomers for making aliphatic homopolymers and copolymers may be selected from the group consisting of, but are not limited to, lactic acid, lactide (including L-, D-, meso and L, D mixtures), glycolic acid, glycolide, e-caprolactone, p-dioxanone, trimethylene carbonate, polyoxaesters, d-valerolactone, b-butyrolactone, e-decalactone, 2,5-diketomorpholine, pivalolactone, a,a-diethylpropiolactone, ethylene carbonate, ethylene oxalate, 3-methyl-1,4-dioxane-2,5-dione, 3,3-diethyl-1,4-dioxan-2,5-dione, g-butyrolactone, 1,4-dioxepan-2-one, 1,5-dioxepan-2-one, 6,6-dimethyl-dioxepan-2-one and 6,8-dioxabicycloctane-7-one. Preferably, the biodegradable polymers are selected from polylactic acid (PLA), polyglycolic acid (PGA), polycaprolactone (PCL), polydioxanone (PDO), trimethylene carbonate (TMC), polyvinyl alcohol (PVA), polyoxaesters, copolymers or blends thereof.

The scaffold can take any form or shape known to those experts in the art. For example it could be in the shape of a solid rod, a cylinder, a pouch, a hollow tube, a sheet or a fibrous network. It can also have a wide range of porosities depending on the required release kinetics. In one embodiment, a scaffold could have a uniform porosity across its three-dimensional matrix and in another it could possess a gradient of porosities.

### The Pharmaceutical Agent

The pharmaceutical agent can be anything that provides a pharmacological response in the patient. It can be either a large or a small molecule. It can be a protein complex. It can be a peptide. It can be a growth factor. And it can be a combination of one or more of the foregoing categories. Ideally, the agent is one that will improve the ability of the transplanted cell(s) or organoid(s) in the scaffold to survive in vivo.

When considering the disease of diabetes mellitus, and assuming the cells and/or organoids in the scaffolds are insulin-producing islets or another type of insulin-producing cell, the pharmaceutical agent is preferably one which will improve the vasculature of the transplant site, modulate the patient's immune system which might otherwise cause the patient's body to reject the transplant, and/or improve the ability of the cells and/or organoids to grow, thrive, and/or differentiate.

Such pharmaceutical compounds can be growth factors, extracellular matrix proteins, and biologically relevant peptide fragments, such as but not limited to members of TGF-b family, including TGF-b1, 2, and 3, bone morphogenic proteins (BMP-2, -3, -4, -5, -6, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, nicotinamide, glucagon-like peptide-I and II, exendin-4, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, antibodies, mimetobodies, MAPK inhibitors, and combinations thereof.

### The Process of Incorporation

The incorporation process utilizes an organic solvent, preferably an alcohol or ether with a relatively high freezing and melting point and a high vapor pressure, to help molecules of the bioactive agent infiltrate the three-dimensional matrix of the scaffold. After the bioactive agent is impregnated into the scaffold matrix, the alcohol or ether is quickly frozen and lyophilized in order to minimize the scaffold's exposure time to the solvent.

The solvent can be any of a number of solvents, such as tertiary butanol. The solvent, and the concentration of the solvent, must be selected with knowledge of both the scaffold and the pharmaceutical agent itself. For proper selection with regard to the scaffold, the solvent must not adversely affect the scaffold material. Further, the concentration must be selected according to the porosity, or density, of the scaffold. As the scaffold density increases, or as the scaffold porosity decreases, the solvent concentration must be increased to overcome the properties related to the hydrophobicity and surface tension of the scaffold.

In a preferred embodiment, the organic solvent used to impregnate the scaffold is one which does not adversely affect either the scaffold or the pharmaceutical compound. As such, it is important that, after the step of incorporating the pharmaceutical compound into the scaffold has been completed, the solvent is completely, or nearly completely, removed.

For those pharmaceutical compounds which are not susceptible to adverse consequences by organic solvents, such as, for example, relatively small molecular weight compounds like p38 kinase inhibitors, it is possible to use up to one hundred percent concentrations of such solvents. It is important, however, that such solvents be selected such that they can be completely removed by, for example, lyophilization. In such case, the solvents will be selected that have relatively high freezing points.

For those pharmaceutical compounds which are susceptible to adverse consequences by organic solvents, such as, for example, relatively high molecular weight molecules like, for example, the protein VEGF or a monoclonal antibody, it is not possible to use solvents in high concentrations without losing activity. Thus, in another preferred embodiment, the organic solvent is in an aqueous solution with the solvent concentration at least about 1%, more preferably at least about 3%, and most preferably at least about 6%. The upper level is dependent on the pharmaceutical compound itself and should be set just below that limit which would cause the pharmaceutical compound to denature. This concentration is enough to overcome the hydrophilic/hydrophobic barrier between the surface of a nonwoven foam composite scaffold and the drug solution. The drug co-solvent solution composition can then be pipetted onto the polymer scaffold as desired. The solvent helps drug molecules in the solution to instantaneously infiltrate the surface of the scaffold into its internal matrix. Subsequently, a freeze-drying technique is used to remove the solvents, leaving behind the molecules of the bioactive deposited within the internal walls of the scaffold.

In a preferred embodiment, the organic solvent contains a large enough alkyl group to lower the surface tension of the aqueous drug solution to allow rapid infiltration. Preferably, the organic solvent contains four or more carbon atoms. In another preferred embodiment the alcohol is tertiary butanol which has a melting point of 25.7° C. and a vapor pressure of 40.7 mm Hg at 25° C. which makes it a perfect candidate for the process. Using a solvent possessing three carbons or less requires the addition of large amounts of the solvent to overcome the hydrophobic barrier on the surface of the scaffold, which may lead to a loss of activity in certain therapeutics such as protein-based compounds which may be denatured due to chemical interactions with the alcohol.

In the case of small molecules that are less sensitive to organic solvents, such as p38 kinase inhibitors, the drug can be dissolved directly in sterile filtered t-butanol and a specific volume based on the amount required pipetted onto the polymer scaffold. The solution is readily absorbable into the polymer matrix allowing rapid spread of the drug molecules into the polymer scaffold. The relatively high freezing point of the solvent allows minimal interaction with the polymer scaffold, thereby protecting the scaffold's internal three-dimensional structure.

For those small bioactive molecules that have poor solubility in traditional organic solvents that may themselves dissolve the polymer, traditional incorporation techniques may not be applicable. For these compounds, the molecules can be dissolved in 100% of the solvent or in a co-solvent system according to the embodiments of this patent.

Pharmaceutical compounds, in general, specifically those with complex sensitive three-dimensional structures, may be aseptically incorporated into pre-sterilized scaffolds using the methods herein. In this method, the scaffold may be fabricated and sterilized via traditional techniques. The pharmaceutical compounds may be separately sterilized by, for example, sterile filtration. In this way, the pharmaceutical compound activities will remain unaffected by the sterilization of the scaffold. After both the scaffold and the pharmaceutical compound have been prepared and sterilized, they may be aseptically combined using the incorporation techniques described herein to produce a scaffold containing the proper amount of active pharmaceutical.

In the method of sterile filtration, pharmaceutical compounds may be dissolved in, for example, a sterile solution of about 0.1% bovine serum albumin or any other stabilizing protein in phosphate buffered saline or any other buffered medium, in the case of relatively large proteins, or in solvent alone, in the case of relatively small molecular weight pharmaceutical agents. The solvent may then be filtered and incorporated into a scaffold in a ratio that is inversely proportional to the porosity of the scaffold but preferably not higher than 10% v/v. A volume of the solution containing the amount of protein required may then be pipetted aseptically onto the scaffold followed by aseptic lyophilization to remove all solvents.

Following the sterile incorporation of a pharmaceutical agent into a biocompatible scaffold according to the methods of the present invention, the scaffold can optionally be seeded with mammalian cells prior to implantation into a host. The mammalian cells may include but not be limited to bone marrow cells, stromal cells, mesenchymal stem cells, embryonic stem cells, umbilical cord blood cells, umbilical Wharton's jelly cells, blood vessel cells, amniotic fluid cells, spleen cells, precursor cells derived from adipose tissue, islets, beta cells, pancreatic ductal progenitor cells, Sertoli cells, peripheral blood progenitor cells, stem cells isolated from adult tissue, oval cells, and genetically transformed cells or a combination of the above cells. The cells can be seeded on the scaffolds for a short period of time (less than one day) just prior to implantation or cultured for longer time periods (greater than one day) to allow for cell proliferation and extracellular matrix synthesis within the seeded scaffold prior to implantation.

The site of implantation is dependent on the diseased or injured tissue that requires treatment. For example, for treatment of a disease such as diabetes mellitus, the cell-seeded scaffold may be placed in a clinically convenient site, such as the subcutaneous space, mesentery, peritoneum, or the omentum. In this particular case, the composite scaffold will act as a vehicle to entrap the administered islets in place after in vivo transplantation into an ectopic site.

### EXAMPLES

The following examples are illustrative of the principles and practice of the invention and are not intended to limit the scope of the invention.

In the examples, the polymers and monomers were characterized in chemical composition and purity (NMR, FTIR), thermal analysis (DSC) and molecular weight by conventional analytical techniques.

Inherent viscosities (I.V., dL/g) of the polymers and copolymers were measured using a 50 bore Cannon-Ubbelhode dilution viscometer immersed in a thermostatically controlled water bath at 30° C. utilizing chloroform or hexafluoroisopropanol (HFIP) as the solvent at a concentration of 0.1 g/dL.

In these examples certain abbreviations are used. These include PCL to indicate polymerized ε-caprolactone; PGA to indicate polymerized glycolide; and PLA to indicate polymerized (L) lactide. Additionally, the ratios in front of the copolymer identification indicate the respective mole percentages of each constituent.

### Example 1

### Fabrication of a Foam Scaffold

The polymer used to manufacture the foam component was a 35/65 PCL/PGA copolymer produced by Birmingham Polymers Inc. (Birmingham, Ala.), with an I.V. of 1.45 dL/g. A 5/95 weight ratio of 35/65 PCL/PGA in 1,4-dioxane solvent was weighed out. The polymer and solvent were placed into a flask, which in turn was put into a water bath and stirred for 5 hours at 70° C. to form a solution. The solution then was filtered using an extraction thimble (extra coarse porosity, type ASTM 170-220 (EC)) and stored in a flask.

A laboratory scale lyophilizer, or freeze dryer, (Model Duradry, FTS Kinetics, Stone Ridge, N.Y.), was used to form the foam scaffold. The polymer solution was added into a 4-inch by 4-inch aluminum mold to a height of 2 mm. The mold assembly then was placed on the shelf of the lyophilizer and the freeze dry sequence begun. The freeze drying sequence used in this example was: 1) −17° C. for 60 minutes, 2) −5° C. for 60 minutes under vacuum 100 mT, 3) 5° C. for 60 minutes under vacuum 20 mT, 4) 20° C. for 60 minutes under vacuum 20 mT.

After the cycle was completed, the mold assembly was taken out of the freeze dryer and allowed to degas in a vacuum hood for 2 to 3 hours. The foam scaffold was stored under nitrogen. The pore size of this composite scaffold was determined using mercury porosimetry analysis. The range of pore size was 1-300 mm with a median pore size of 45 mm.

### Example 2

### Forming a Composite Foam Nonwoven Scaffold

A needle-punched nonwoven mat (2 mm in thickness) composed of a 90/10 PGA/PLA fiber was made as described below. A copolymer of PGA/PLA (90/10) was melt-extruded into continuous multifilament yarn by conventional methods of making yarn and subsequently oriented in order to increase strength, elongation and energy required to rupture. The yarns comprised filaments of approximately 20 microns in diameter. These yarns were then cut and crimped into uniform 2-inch length to form 2-inch staple fibers.

A dry lay needle-punched nonwoven mat was then prepared utilizing the 90/10 PGA/PLA copolymer staple fibers. The staple fibers were opened and carded on standard nonwoven machinery. The resulting mat was in the form of webbed staple fibers. The webbed staple fibers were needle punched to form the dry lay needle-punched, fibrous nonwoven mat.

The mat was scoured with isopropanol for 60 minutes, followed by drying under vacuum.

The polymer used to manufacture the foam component was a 35/65 PCL/PGA copolymer produced by Birmingham Polymers Inc. (Birmingham, Ala.), with an I.V. of 1.45 dL/g. A 0.5/99.5 weight ratio of 35/65 PCL/PGA in 1,4-dioxane solvent was weighed out. The polymer and solvent were placed into a flask, which in turn was put into a water bath and stirred for 5 hours at 70° C. to form a solution. The solution then was filtered using an extraction thimble (extra coarse porosity, type ASTM 170-220 (EC)) and stored in a flask.

A laboratory scale lyophilizer, or freeze dryer, (Model Duradry, FTS Kinetics, Stone Ridge, N.Y.), was used to form the composite scaffold. Approximately 10 ml of the polymer solution was added into a 4-inch by 4-inch aluminum mold to cover uniformly the mold surface. The needle-punched nonwoven mat was immersed into the beaker containing the rest of the solution until fully soaked and was then placed in the aluminum mold. The remaining polymer solution was poured into the mold so that the solution covered the nonwoven mat and reached a height of 2 mm in the mold. The mold assembly then was placed on the shelf of the lyophilizer and the freeze-drying sequence begun. The freeze drying sequence used in this example was: 1) −17° C. for 60 minutes, 2) −5° C. for 60 minutes under vacuum 100 mT, 3) 5° C. for 60 minutes under vacuum 20 mT, 4) 20° C. for 60 minutes under vacuum 20 mT.

After the cycle was completed, the mold assembly was taken out of the freeze drier and allowed to degas in a vacuum hood for 2 to 3 hours. The composite scaffolds then were stored under nitrogen.

### Example 3

### Incorporation of a p38 Kinase Inhibitor into a Composite Scaffold and Testing of Total Drug Content within the Scaffold

A foam nonwoven composite sheet was prepared as indicated in example 2. Cylindrical scaffolds with a diameter of 8 mm were punched out using dermal biopsy punches and terminally sterilized via EtO sterilization technique. In a sterile hood and using aseptic techniques, the scaffolds were placed each separately in the wells of a sterile 24-well plate. A solution of 1 mg/ml of a p38 kinase inhibitor, JNJ 3026582 (4-[4-(4-fluorophenyl)-1-(3-phenylpropyl)-5-(4-pyridinyl)-1H-imidazol-2-yl]-3-butyn-1-ol) in tertiary butanol was prepared and sterile filtered through a 0.2-micron filter into a sterile polypropylene tube. An aliquot of 30 μl of said solution was then aseptically pipetted onto each scaffold. Scaffolds were then aseptically freeze-dried to remove solvent.

Assessing the total content of p38 kinase inhibitor in the scaffold: Three scaffolds prepared as indicated above were each placed in a 2 ml glass vial containing 0.5 ml of methanol. The vial was then sealed and placed on a shaker overnight to ensure complete release of the drug content from the scaffold into the solvent. The following day the scaffold was removed from the vial and the solution was analyzed via HPLC to determine the total drug content. A 5 μm Zorbax SB-C8 (250×4.6 mm) column was used with a mobile phase composed of 40% acetonitrile, 52% methanol, and 8% water. The flow rate was controlled at 1 ml/min. The injection volume was 10 μl and detection was done at 240 nm. The retention time of the p38 kinase inhibitor was 8.0 minutes. A stock solution was prepared by dissolving the drug in pure methanol and stored refrigerated in an amber flask. The standard solutions were prepared by serial dilution of the stock solution. The calibration curve was found to be linear in the range of 5-100 μg/ml. Linearity and reproducibility were assessed by duplicate injections of four standards.

Result: The total content of p38 kinase inhibitor on each of the tested scaffolds was as follows: 29, 29, and 29.5 micrograms. This shows the accuracy of the incorporation method, as the theoretical value of the drug deposited on each scaffold was 30 micrograms.

### Example 4

### Incorporation of p38 Kinase Inhibitor into a Foam Scaffold and Testing Total Drug Content

In a similar fashion to that described in Example 3, p38 kinase inhibitor was incorporated into the foam scaffolds prepared as described above in example 1. Similarly the total drug content on each of three foam scaffolds was determined. The results were as follows: 29.5, 30, and 30.5 micrograms.

### Example 5

### Incorporation of VEGF-121 into a Foam Nonwoven Composite Scaffold

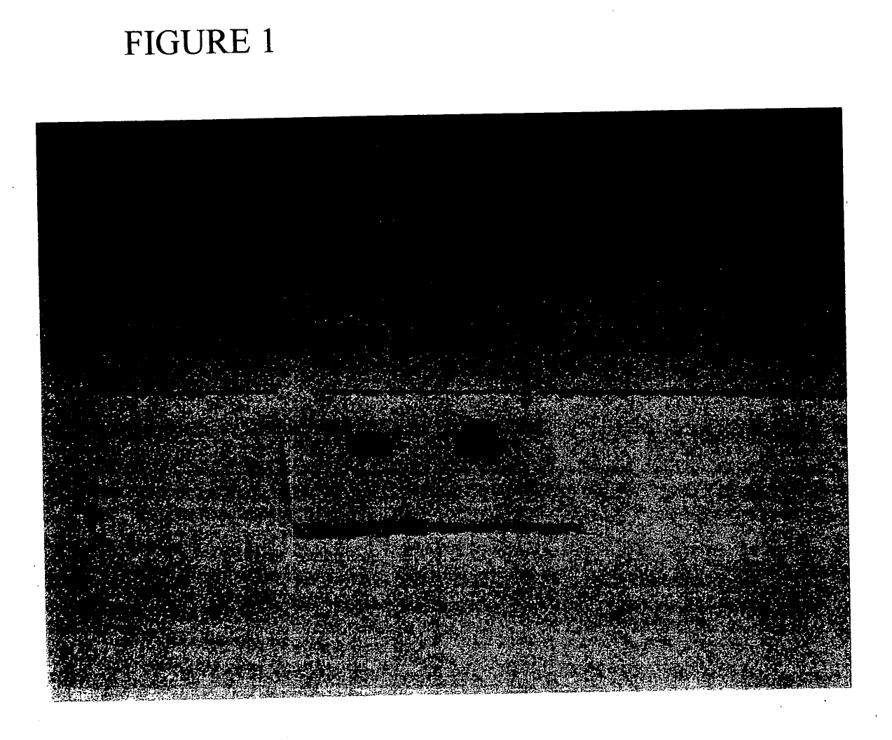

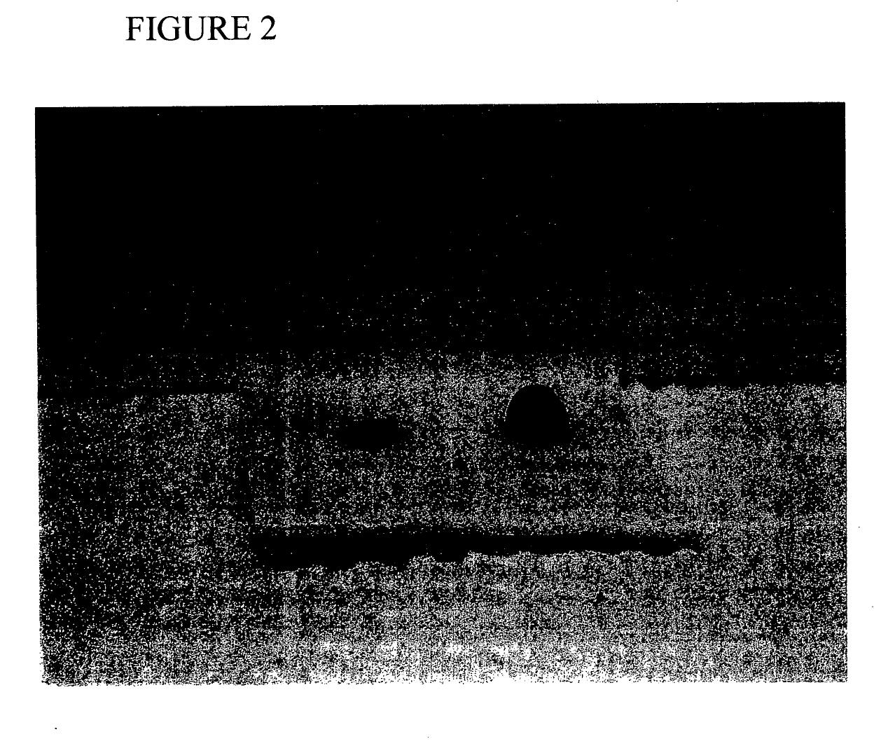

Determining the minimum amount of alcohol required for the incorporation: A solution of 2.1 mg/ml VEGF-121 in PBS (vascular endothelial growth factor, obtained from Scios Inc.) was diluted to 0.5 mg/ml with a co-solvent system of PBS and tertiary butanol so that the total tertiary butanol to PBS volume to volume ratio in the final solution was 0% or 1% or 2% or 5% or 10% or 20% or 30%. A 30 μl drop of each of the previous solutions was pipetted onto a foam nonwoven composite scaffold with an 8 mm diameter fabricated as previously described. The time required for full infiltration of each of the solution into the scaffold was assessed. Solutions with 10%, 20%, and 30% ratios instantaneously infiltrated their respective scaffolds. On the other hand, solutions with 0%, 1%, 2%, and 5% ratios did not. A similar solution was prepared with a ratio of 6% and was tested in a similar manner. This is exemplified in FIGS. 1 and 2 where the effect of this concentration of t-butanol is compared to a solution that does not contain t-butanol. FIG. 1 shows the effect after ten seconds, and FIG. 2 shows the effect after thirty minutes. Other alcohols, such as methanol, ethanol, and iso-propanol, were tested at the same alcohol to PBS ratio but did not achieve infiltration at the 6% ratio. They did, however, achieve infiltration at much higher ratios, which negatively influenced the activity of VEGF-121.

Assessing the total content of VEGF-121 incorporated into a composite scaffold: A 0.5 mg/ml solution of VEGF-121 in a co-solvent system of PBS and tertiary butanol was prepared with the tertiary butanol to PBS ratio being 6%. An aliquot of 20 μl of said solution was pipetted onto each of three composite scaffolds. Within 10 seconds, VEGF solutions completely infiltrated the scaffolds, which were then frozen and the solvents sublimed as described before. A micro BCA protein assay kit (manufactured by PIERCE, Illinois) was used to assess the total VEGF-121 content in each of the scaffolds.

Standard solutions were prepared by serial dilution of a stock solution of 2.1 mg/ml. The calibration curve was found to be linear in the range of 0.5-20 μg/ml, which correlated well with the values indicated by the kit protocol. Each of the three scaffolds was placed in a polypropylene tube that contained 1 ml of PBS and shaken overnight at 37° C. The test results showed that the scaffold incorporated on average 99.5% of the available amount of VEGF-121, indicating the high efficiency of the process.

### Example 6

### In-Vivo Testing of the Activity of VEGF-121 Incorporated into a Composite Scaffold

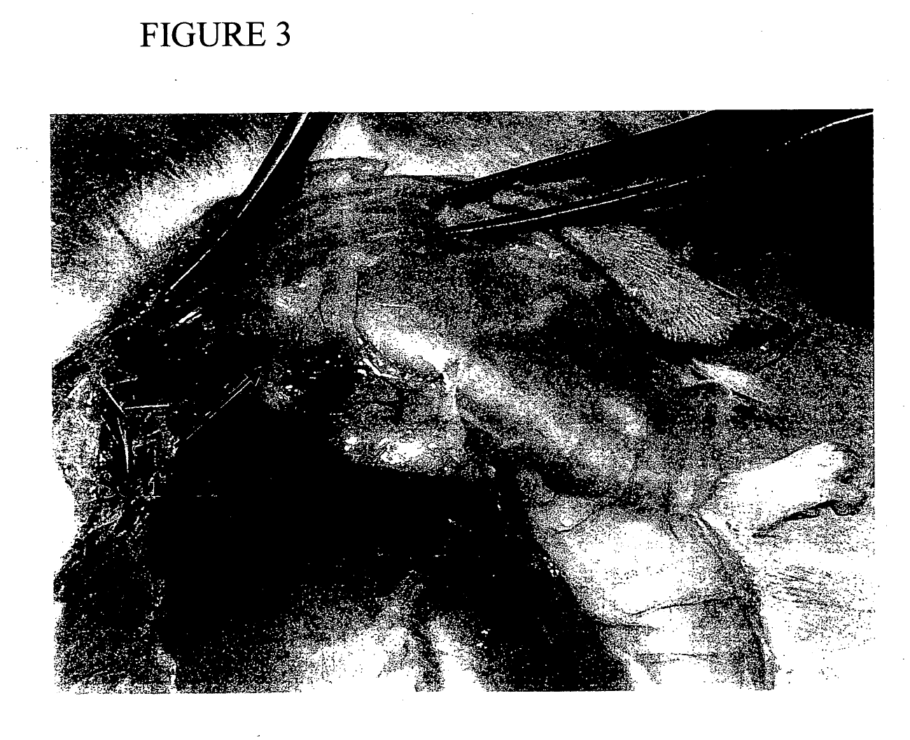

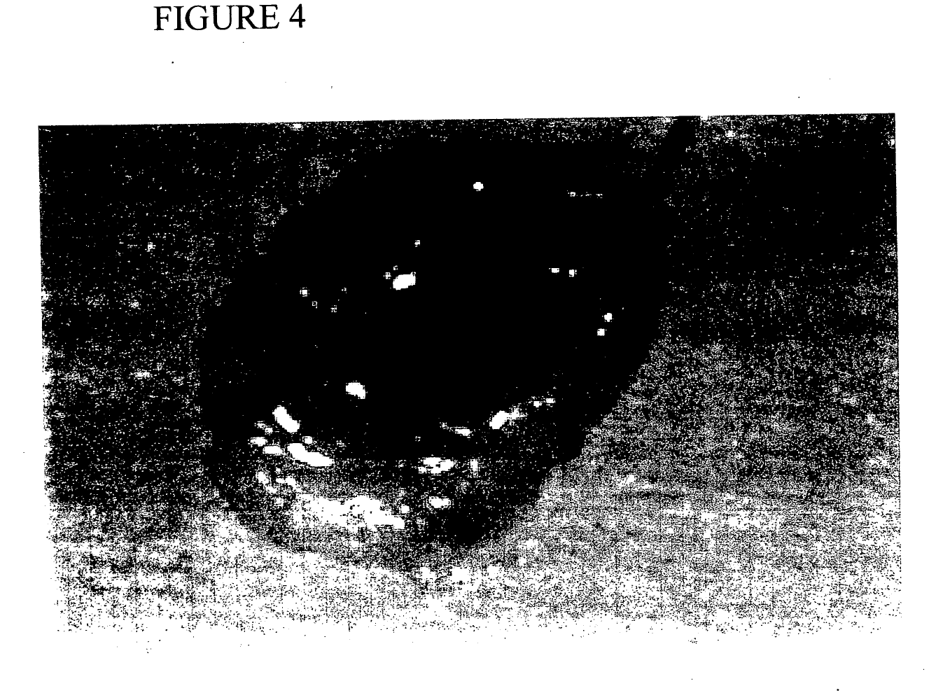

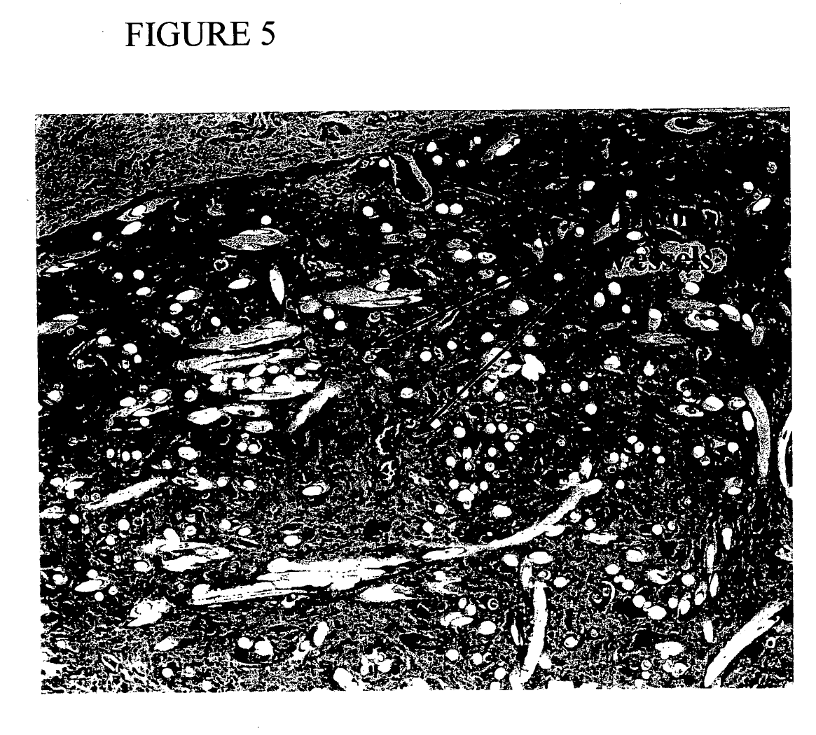

An amount of 10 μg of VEGF-121 was incorporated aseptically into each of four sterile foam nonwoven composite scaffolds as indicated in the examples above. Another four composite scaffolds received blank solutions as controls. Two Spargue-Dawley rats weighing approximately 200 gram were used as recipients. Animals were anesthesized with isoflorane and the surgical site shaved and prepped with betadine and alcohol. A dorsal midline incision was made and control and treated scaffolds were placed in four subcutaneous locations; the right and left cranial and caudal areas. Each animal received two scaffolds containing VEGF-121 and two blank scaffolds. The animals were sacrificed at 14 days following device implantation. Scaffolds were photographed in situ (FIG. 3) followed by removal (FIG. 4) and fixation in 10% buffered formalin for histological evaluation. The results of the histological evaluation (FIG. 5) demonstrated a high level of vascularization through the scaffolds that received VEGF-121, indicating that the incorporation process did not have a negative impact on the bioactivity of VEGF-121 in vivo.

### Example 7

### Evaluating the Effect of Tertiary Butanol and Lyophilization on the Bioactivity of VEGF-121

Human umbilical vein endothelial cells (HUVEC; Cambrex BioScience, Walkersville, Md.) were maintained in log phase in optimal medium (EGM-2 fully constituted medium; Cambrex BioScience). On the day of assay, cells were trypsinized to detach, counted, adjusted to a concentration of 40,000 cells/ml in DMEM containing 5% FCS (fetal calf serum), and plated at a density of 4000 cells/well (100 μl) in a 96-well flat-bottom plate.

A stock solution of VEGF 121 was diluted to 1 μg/ml in PBS with 0.1% BSA. Aliquots of 1 ml each, one with no further treatment and one receiving 10% (v/v) t-butanol, were frozen, lyophilized to dryness, and then reconstituted to 1 ml each (11 g/ml) with DMEM containing 5% FCS. Two-fold serial dilutions in 100 μl volumes of DMEM/FCS were added in triplicate to assay plates containing HUVEC as plated above. An original sample of VEGF 121, not lyophilized but diluted similarly, served as a positive control. Sample wells of cells receiving no added growth factor (DMEM/FCS alone) served as a background negative control.

Cells were cultured for a total of 72 hours with a pulse of 1 μCi ³H-thymidine per well during the last 24 hours. To terminate the assay, 150 μl of medium was aspirated from each well, replaced with 150 μl of water, and plates were frozen overnight at −80° C. Plates were thawed and harvested onto filters using a Packard Filtermate Harvester prior to counting on a Packard TopCount NXT™ scintillation counter.

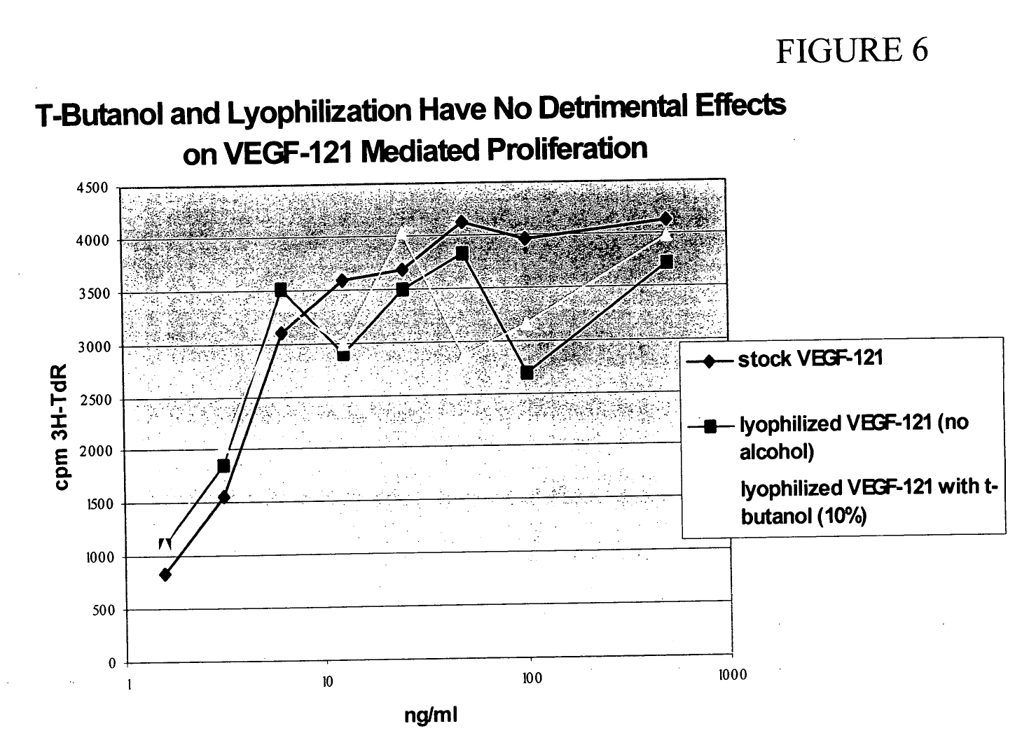

The results of the biological assay as represented in (FIG. 6) show that the biological activity of VEGF-121 was not negatively affected by exposure to the indicated levels of tertiary butanol nor by the lyophilization process.

### Example 8

### Evaluating the Bioactivity of VEGF-121 after its Recovery from a Composite Foam Nonwoven Scaffold

Human umbilical vein endothelial cells (HUVEC; Cambrex Bio Science) were maintained in log phase in optimal medium (EGM-2 fully constituted medium; Cambrex Bio Science). On the day of assay, cells were trypsinized to detach, counted, adjusted to a concentration of 40,000 cells/ml in DMEM containing 5% FCS, and plated at a density of 4000 cells/well (100 μl) in a 96-well flat-bottom plate.

A 0.5 mg/ml solution of VEGF-121 in a co-solvent system of PBS/0.1% BSA and tertiary butanol was prepared with the tertiary butanol to PBS/BSA ratio being 10%. A volume of 20 μl of said solution (1 μg) was pipetted onto each of three foam nonwoven composite scaffolds (8 mm diameter), fabricated as previously described. VEGF solutions completely infiltrated the scaffolds instantaneously. Another three composite scaffolds received blank solutions as controls (PBS/BSA with tertiary butanol co-solvent alone). Scaffolds were then frozen and solvents were sublimed as previously described. Each scaffold was eluted with continuous shaking in a total volume of 1 ml medium (DMEM with 5% FCS) at 37° C. Eluted volumes were treated as hypothetical 1 μg/ml solutions and compared to a known standard stock of VEGF 121 (1 μg/ml). Two-fold serial dilutions in 100 μl volumes of DMEM/FCS were added in triplicate to assay plates containing HUVEC as plated above. Sample wells of cells receiving no added growth factor (DMEM/FCS alone) served as a background negative control.

Cells were cultured for a total of 72 hours with a pulse of 1 μCi ³H-thymidine per well during the last 24 hours. To terminate the assay, 150 μl of medium was aspirated from each well, replaced with 150 μl of water, and plates were frozen overnight at −80° C. Plates were thawed and harvested onto filters using a Packard Filtermate Harvester prior to counting on a Packard TopCount NXT™ scintillation

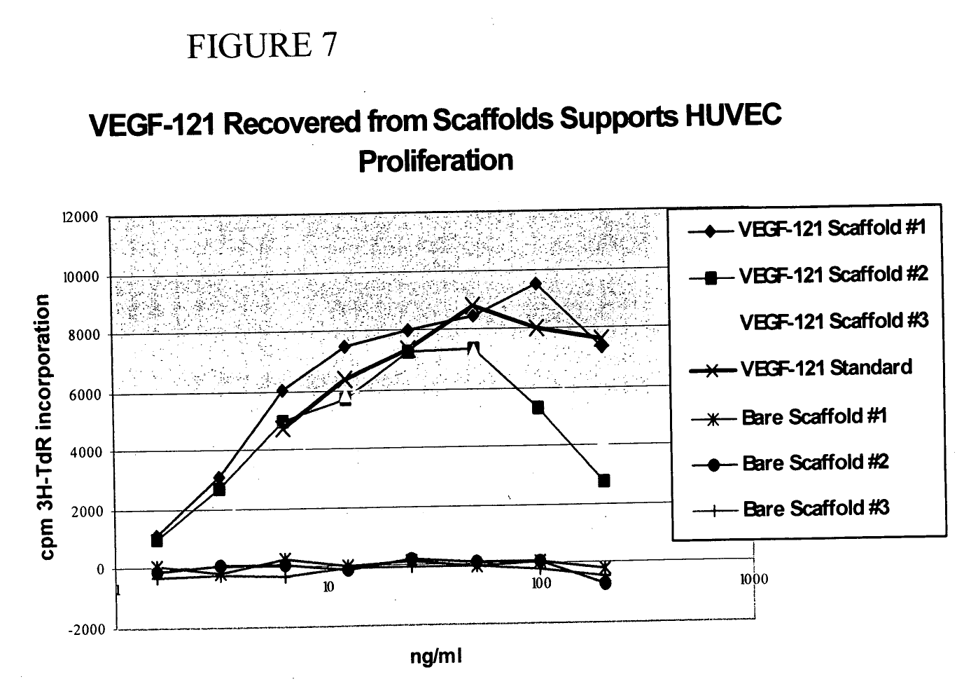

The results represented in (FIG. 7) show that VEGF-121 recovered from scaffolds expressed similar bioactivity to that of a stock solution, indicating that the incorporation method sustained the activity of the growth factor.

What is claimed is:

1. A method of incorporating a pharmaceutical agent into a scaffold, the method comprising:
a. selecting a scaffold,
b. selecting a pharmaceutical agent;
c. dissolving the pharmaceutical agent into a mixture containing an organic solvent;
d. bringing the solution containing the pharmaceutical agent and the organic solvent into contact with the scaffold; and
e. removing a portion of the solvent.

2. The method of claim 1 wherein the scaffold is a composite scaffold.

3. The method of claim 2 wherein the composite scaffold has a foam element.

4. The method of claim 3 wherein the foam element is manufactured from a 0.5% polymer solution.

5. The method of claim 1 wherein the scaffold is made by lyophilization.

6. The method of claim 1 wherein the scaffold is made from aliphatic polyesters.

7. The method of claim 6 wherein the aliphatic polyesters are homopolymers.

8. The method of claim 6 wherein the aliphatic polyesters are copolymers.

9. The method of claim 6 wherein the aliphatic polyesters are manufactured from monomers selected from the group consisting of; lactic acid, lactide, glycolic acid, glycolide, ε-caprolactone, p-dioxanone, trimethylene carbonate, polyoxaesters, d-valerolactone, b-butyrolactone, e-decalactone, 2,5-diketomorpholine, pivalolactone, a,a-diethylpropiolactone, ethylene carbonate, ethylene oxalate, 3-methyl-1,4-dioxane-2,5-dione, 3,3-diethyl-1,4-dioxan-2,5-dione, g-butyrolactone, 1,4-dioxepan-2-one, 1,5-dioxepan-2-one, 6,6-dimethyl-dioxepan-2-one and 6,8-dioxabicycloctane-7-one.

10. The method of claim 1 wherein the scaffold is made from materials selected from the group consisting of polylactic acid, polyglycolic acid, polycaprolactone, polydioxanone, trimethylene carbonate, polyvinyl alcohol, polyoxaesters, copolymers or blends thereof.

11. The method of claim 1 wherein the scaffold is made from a polyglycolic acid—polycaprolactone copolymer.

12. The method of claim 1 wherein the pharmaceutical agent is one that is affected by sterilization.

13. The method of claim 1 wherein the pharmaceutical agent is one that is denatured by organic solvents.

14. The method of claim 1 wherein the pharmaceutical agent is a growth factor.

15. The method of claim 1 wherein the pharmaceutical agent is an extracellular matrix protein.

16. The method of claim 1 wherein the pharmaceutical agent is a biologically relevant peptide fragment.

17. The method of claim 1 wherein the pharmaceutical agent is a biologically relevant peptide fragment of the TFG-b family.

18. The method of claim 17 wherein the peptide fragment is selected from the group consisting of TGF-B1, 2 and 3.

19. The method of claim 1 wherein the pharmaceutical agent is a bone morphogenic protein.

20. The method of claim 19 wherein the bone morphogenic protein is selected from the group consisting of BMP-2, -3, -4, -5, -6, -11, -12, and -13.

21. The method of claim 1 wherein the pharmaceutical agent is selected from the group consisting of: fibroblast growth factors-1 and -2, platelet-derived growth factors-AA, and -BB, platelet rich plasma, insulin growth factors IGF-I, II, growth differentiation factors GDF-5, -6, -8, -10, -15, vascular endothelial cell-derived growth factor VEGF, pleiotrophin, endothelin, nicotinamide, glucagon-like peptide-I and II, exendin-4, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins, antibodies, mimetobodies, MAPK inhibitors, and combinations thereof

22. The method of claim 1 wherein the organic solvent is an alcohol.

23. The method of claim 1 wherein the organic solvent is an ether.

24. The method of claim 22 wherein the alcohol has four or more carbon atoms.

25. The method of claim 24 wherein the alcohol is t-butanol.

26. The method of claim 1 wherein the organic solvent is used in a concentration of at least about 1%.

27. The method of claim 26 wherein the organic solvent is used in a concentration of at least about 3%.

28. The method of claim 27 wherein the organic solvent is used in a concentration of at least about 6%.

29. The method of claim 1 wherein the organic solvent is used in an amount that is not sufficient to denature the pharmaceutical agent.

30. The method of claim 1 wherein before the pharmaceutical agent and the scaffold are brought into contact with each other they are separately sterilized.

31. The method of claim 30 wherein in the step of bringing the pharmaceutical agent into contact with the scaffold such is done aseptically.

32. The method of claim 1 wherein all of the solvent is removed.

33. The method of claim 1 wherein the solvent is removed by lyophilization.

34. The method of claim 1 wherein the pharmaceutical agent is selected from the group consisting of VEGF-121 and a p38 kinase inhibitor or combinations thereof.

35. A method of transplanting mammalian cells into a patient, the method comprising:
a. selecting a scaffold,
b. selecting a pharmaceutical agent;
c. dissolving the pharmaceutical agent into a mixture containing an organic solvent;
d. bringing the solution containing the pharmaceutical agent and the organic solvent into contact with the scaffold;
e. removing a portion of the solvent;
f. seeding the scaffold with mammalian cells; and
g. transplanting the scaffold into the patient.

36. A method of transplanting mammalian cells into a patient, the method comprising:
a. selecting a scaffold,
b. selecting a pharmaceutical agent;
c. dissolving the pharmaceutical agent into a mixture containing an organic solvent;
d. bringing the solution containing the pharmaceutical agent and the organic solvent into contact with the scaffold;
e. removing a portion of the solvent;
f. transplanting the scaffold into the patient; and
g. seeding the scaffold with mammalian cells.

37. A scaffold that has been impregnated with a pharmaceutical agent using a process comprising:
a. selecting a scaffold,
b. selecting a pharmaceutical agent;
c. dissolving the pharmaceutical agent into a mixture containing an organic solvent;
d. bringing the solution containing the pharmaceutical agent and the organic solvent into contact with the scaffold; and
e. removing a portion of the solvent.

38. A process of manufacturing a sterile scaffold containing a pharmaceutical compound comprising:
a. sterilizing the scaffold;
b. sterilizing the pharmaceutical compound; and
c. aseptically incorporating the pharmaceutical compound into the sterile scaffold.
